(19) United States
(12) Patent Application Publication
(10) Pub. No.: US 2009/0292363 A1
(43) Pub. Date: Nov. 26, 2009

(54) INTERVERTEBRAL PROSTHESIS

(75) Inventors: Michael Goldfarb, Franklin, TN (US); Skyler A. Dalley, Nashville, TN (US)

Correspondence Address: DARBY & DARBY P.C.;CHURCH STREET STATION, P.O. BOX 770, NEW YORK, NY, 10008-0770, US

(73) Assignee: Vanderbilt University, Nashville, TN (US)

(21) Appl. No.: 12/470,690

(22) Filed: May 22, 2009

Related U.S. Application Data
(60) Provisional application No. 61/055,522, filed on May 23, 2008.

(51) Int. Cl. A61F 2/44

(52) U.S. Cl. 623/17.16; 623/17.13

(57) ABSTRACT

A prosthesis for replacing a native disc between first and second adjacent vertebral bodies. The prosthesis includes a compliant element having a first composition and a geometry for providing a plurality of element stiffnesses for the compliant element substantially matching spatial stiffnesses of the native disc. The prosthesis also includes an upper plate of the first or a second composition, the upper plate having opposed inner and outer surfaces, the upper plate inner surface having a first retaining structure for affixing a position of the first end of the compliant element, and a lower plate of the first or a second composition, the lower plate having opposed inner and outer surfaces, the lower plate inner surface having a second retaining structure for affixing a position of the second end of the compliant element.

### CROSS REFERENCE TO RELATED APPLICATIONS

This application claims the benefit of Provisional Application Ser. No. 61/055,522 entitled “INTERVERTEBRAL PROSTHESIS”, filed May 23, 2008, which is herein incorporated by reference in its entirety.

### FIELD OF THE INVENTION

The invention relates to spinal column prostheses, and more particularly to intervertebral spinal disc replacement prostheses.

### BACKGROUND

There is increasing interest in using functional intervertebral disc replacement procedures (i.e., joint arthroplasty) to replace conventional spinal fusion procedures. As a result, many types and configurations of prosthetic spinal discs have been proposed and used for joint arthroplasty. Prosthetic spinal discs can generally be classified as either a viscoelastic type or a kinematic type.

Viscoelastic prosthetic discs are typically constructed of a silicone or other polymer-comprising material that substantially reproduces the spatial compliance of the biological or native spinal disc in a generally homogeneous manner. The primary difficulty with the use of these discs, however, is the relatively short lifespan of such materials under in vivo loading conditions. In particular, conventional viscoelastic discs are susceptible to creep and material flow. Additional difficulties typically include the inability to tailor the spatial properties of the material to match the heterogeneous nature of a native disc and the difficulty in bonding such materials to bone. As such, the lifespan of conventional viscoelastic discs is typically a substantial issue.

The second type of prosthetic disc design, the kinematic design, typically utilizes a variation on a ball or saddle joint to replace the native disc, typically constructed from metals or a combination of metals and plastics. Such materials, unlike viscoelastic materials, generally provide acceptable life spans. However, kinematic designs typically over-constrain the joint, and thus decrease Joint mobility and increase internal joint loading. Additionally, since such discs are not spatially compliant, they generally lack the shock-absorbing capacity of native discs and decrease the (postural) stability of the joint promoted by the stiffness of the native disc. Therefore, what is needed is a prosthetic disc that has both an acceptable life span and provides acceptable spatial compliance.

### SUMMARY

This Summary is provided to comply with 37 C.F.R. §1.73, presenting a summary of the invention to briefly indicate the nature and substance of the invention. It is submitted with the understanding that it will not be used to interpret or limit the scope or meaning of the claims. In a first embodiment of the invention, a prosthesis is provided for replacing a native disc between first and second adjacent vertebral bodies. The prosthesis includes a compliant element having a first composition and a geometry for providing a plurality of element stiffnesses for the compliant element substantially matching spatial stiffnesses of the native disc. The prosthesis also includes an upper plate of the first or a second composition, the upper plate having opposed inner and outer surfaces, the upper plate inner surface having a first retaining structure for affixing a position of the first end of the compliant element, and a lower plate of the first or a second composition, the lower plate having opposed inner and outer surfaces, the lower plate inner surface having a second retaining structure for affixing a position of the second end of the compliant element.

In a second embodiment of the invention, a method for designing a prosthesis for replacing a native disc between first and second adjacent vertebral bodies is provided. The method includes the step of determining a geometry for a complaint element of a first composition, the geometry providing element stiffnesses for the compliant substantially matching spatial stiffnesses of the native disc, the geometry distributing a force applied to at least one of a first and a second end of the complaint element to a plurality of other portions of the compliant such that a portion of the force distributed to each of the other spring portions under nominal native disc loading conditions is less than an endurance limit of the first composition. The method also includes the step of designing an upper plate of the first or a second composition, the upper plate having opposed inner and outer surfaces, the upper plate inner surface designed to have a first retaining structure for affixing a position of the first end of the compliant element. The method further includes the step of designing a lower plate of the first or a second composition, the lower plate having opposed inner and outer surfaces, the lower plate inner surface designed to have a second retaining structure for affixing a position of the second end of the compliant element.

In a third embodiment of the invention, a prosthesis for replacing a native disc between first and second adjacent vertebral bodies is provided. The prosthesis includes a wave spring having a first composition and a geometry for providing stiffnesses for the spring substantially matching a stiffnesses of the native disc, the geometry distributing a force applied to at least one of a first and a second end of the spring to a plurality of other portions of the spring, and the first composition having an endurance limit greater than a portion of the force distributed to each of the other spring portions under nominal native disc loading conditions. The prosthesis also includes an upper plate of the first or a second composition, the upper plate having opposed inner and outer surfaces, the upper plate inner surface having a first retaining structure for affixing a position of the first end of the spring. The prosthesis further includes a lower plate of the first or a second composition, the lower plate having opposed inner and outer surfaces, the lower plate inner surface having a second retaining structure for affixing a position of the second end of the spring.

### BRIEF DESCRIPTION OF THE DRAWINGS

FIG. 1 shows an exemplary intervertebrate prosthesis according to an embodiment of the invention.

FIG. 2 shows an exemplary intervertebrate prosthesis placed between adjacent vertebrate bodies according to an embodiment of the invention.

FIG. 3 shows an exemplary wire for a wave spring according to an embodiment of the invention.

FIG. 4 shows and exploded view of an exemplary intervertebrate prosthesis according to an embodiment of the invention.

FIG. 5 shows an exemplary intervertebrate prosthesis according to an embodiment of the invention.

FIGS. 6A and 6B show posterior and side views of another exemplary intervertebrate prosthesis according to an embodiment of the invention.

FIG. 7 schematically shows axes of interest with respect to a vertebrate body.

FIG. 8 shows a front view of an elliptical wave spring in accordance with an embodiment of the invention.

FIG. 9 shows a top view of an elliptical wave spring in FIG. 8.

FIG. 10A shows tabulated design constraints and nominal design values for a stainless steel comprising intervertebrate prosthesis for female patients, age 50-59, in accordance with an embodiment of the invention.

FIG. 10B shows tabulated design constraints and nominal design values for a stainless steel comprising intervertebrate prosthesis for male patients, age 50-59, in accordance with an embodiment of the invention.

FIG. 11A shows tabulated design constraints and nominal design values for titanium comprising intervertebrate prosthesis for female patients, age 50-59, in accordance with an embodiment of the invention.

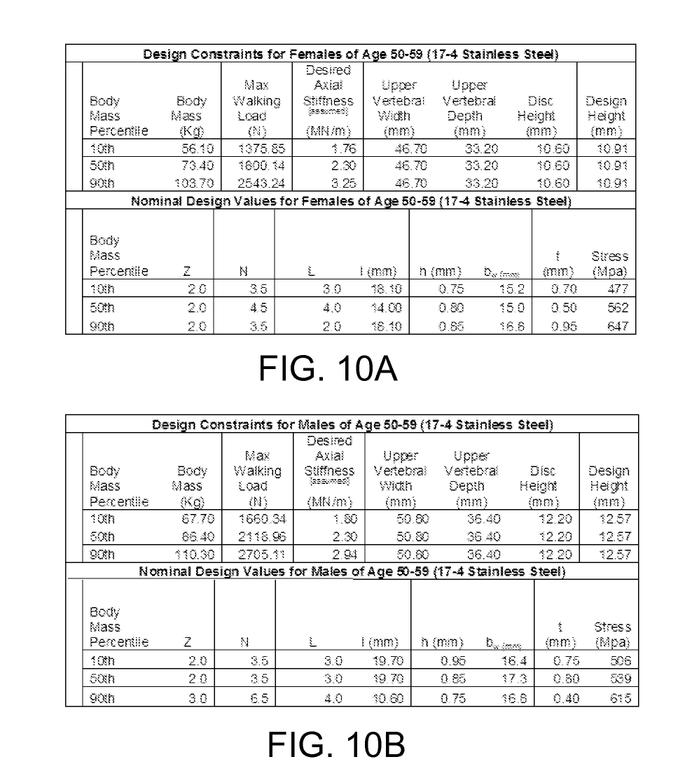

FIG. 10A shows tabulated design constraints and nominal design values for titanium comprising intervertebrate prosthesis for male patients, age 50-59, in accordance with an embodiment of the invention.

### DETAILED DESCRIPTION

The invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The invention is not limited by the illustrated ordering of acts or events, as some acts may occur in different orders and/or concurrently with other acts or events. Furthermore, not all illustrated acts or events are required to implement a methodology in accordance with the invention.

As previously discussed, the major drawbacks with conventional intervertebrate prostheses is that they are typically (1) kinematically constrained with limited or no spatial compliance or (2) provide reasonable spatial compliance but unacceptable life spans of operation. To overcome these problems, embodiments of the invention provide a prosthetic spinal disc including compliant element, such as a spring, constructed from materials providing acceptable life spans. Furthermore, by tailoring the design of the compliant element to account for the maximum stresses typically seen by native discs, an element design can be used such stresses on the compliant can remain under the endurance limit of the materials and provide an acceptable life span. In other words, spinal discs according to the various embodiments of the invention include a compliant element having an acceptable life span (>30 million cycles) and providing stiffnesses close to that of the native disc. For example, the case of human patients, this means trying to match or closely approximate the stiffness of a native disc in terms of compression (˜2.3 MN/m), shear (˜0.26 MN/m), extension (˜1.3 Nm/deg), flexion (˜0.8 Nm/deg), lateral bending (˜1.1 Nm/deg), and torsion (˜2 Nm/deg) (collectively the “spatial stiffnesses”). However, the invention is not limited to solely human patients and can be used for replacement of spinal discs in any organism having spinal discs.

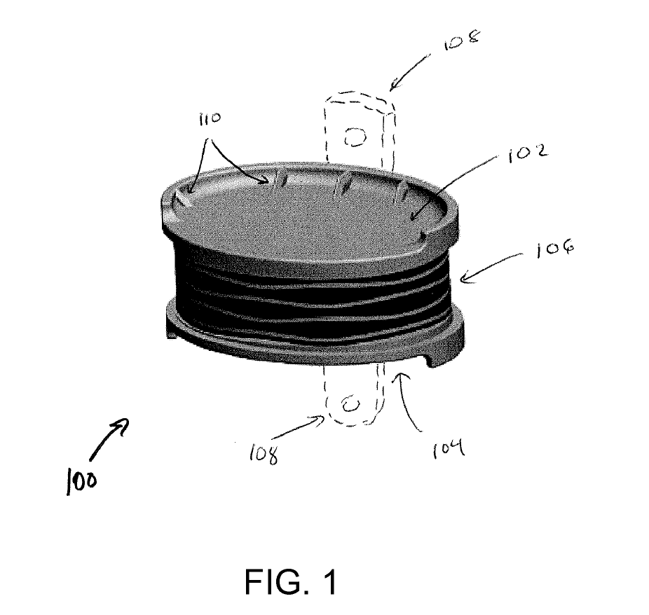

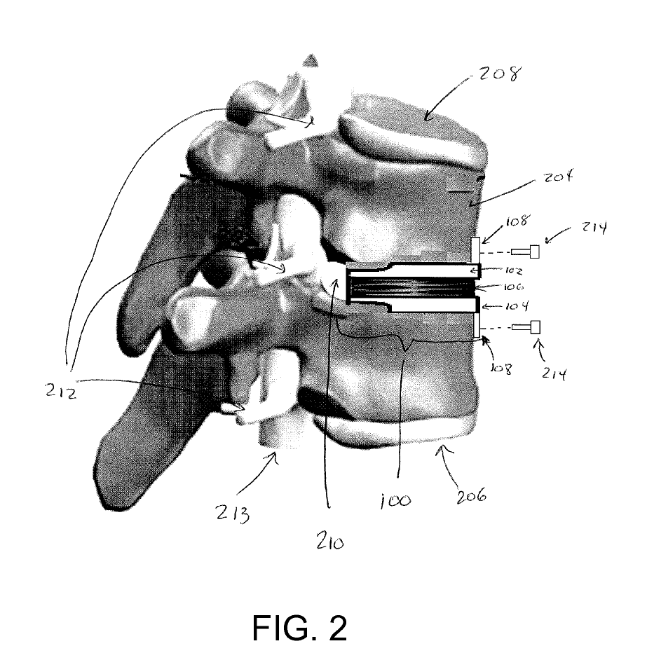

An exemplary embodiment of a intervertebrate prosthesis, according to the invention, is shown in FIG. 1. As shown in FIG. 1, the disc 100 consists of three main parts, which include an upper endplate 102, a lower endplate 104, and a spatially compliant spring 106 which is sandwiched in between the endplates 102, 104. FIG. 2 illustrates how the disc 100 could be positioned between lower 202 and upper 204 vertebrate bodies. Typically, the vertebrate bodies 202, 204 would be separated by native discs 206, 208. However, if a native disc in a region 210 is diseased or otherwise degenerated, the disc 100 would be implanted by removing the damaged native disc material in region 210, inserting and securing the endplates 102, 104 of the disc 100 onto the adjacent vertebral bodies 202, 204, and then compressing and inserting the spring 106 in between the two endplates 102, 104, where the spring 106 would remain in a compressed state. However, the invention is not limited in this regard. For example, disc 100 can be inserted into region 210 and secured to vertebral bodies 202, 204 as a single component. Although disc 100 is illustrated to only extend into a portion of region 210, one of ordinary skill in the art will recognize that the disc 100 can be sized to extend any distance into region 210. One of ordinary skill in the art will recognize that the distance the disc 100 protrudes into region 210 can be limited to ensure that the disc does not contact or damage nerves 212 or spinal cord 213. Furthermore, the disc 100 can generally be sized to any height necessary to restore proper spacing and alignment of the vertebral bodies 202, 204.

In the various embodiments of the invention, the endplates 102, 104 can be secured to vertebral bodies in several ways. For example, as shown in FIG. 1, the endplates 102, 104 can include anchoring features 108 extending from the endplates 102, 104, to attach the endplates 102, 104 to vertebral bodies using fasteners. (e.g., screws, nails, clips, anchors, etc.). For example, as shown in FIG. 2, the endplates 102, 104 can be inserted into region 210 until the anchoring features are adjacent to surface of the vertebral bodies 204, 202. The endplates 102, 104 can then be attached to the vertebral bodies using screws 214, as shown in FIG. 2. However, it is also within the scope of the invention to secure the disc in place without mechanical fasteners, for example with a biocompatible adhesive or osseointegration techniques to attach the endplates 102, 104 to vertebral bodies. “Biocompatible,” as used herein refers to materials which are inert or non-reactive when implanted on or in a biological entity.

In some embodiments, the surface of the endplates 102, 104 can include surface anchoring features designed to hold the disc in place. For example, one or more surface anchors 110, as shown in FIG. 1, can be used. The surface anchors 110 can be configured to drive into the vertebral bodies and form grooves in the vertebral bodies under an initial load. The force exerted by the spring 106 can then retain the surface anchors 110 in the grooves. In other embodiments, at least a portion of the surface of the endplates 102, 104 can be configured to match the contours of the vertebral bodies. Again, the force exerted by the spring 106 can retain the endplates 102, 104 in place. However, the invention is not limited to individual the examples above. In some embodiments, a combination of fasteners, surface anchors, and adhesives can also be used. For example, in FIG. 2, an adhesive material can be inserted between an endplate 102, 104 and adjacent vertebral bodies 202, 204, even though screws 214 are being used.

In the various embodiments of the invention, the spring design can be developed using a mathematical model for the six degrees of freedom of the native disc. The mathematical model can be utilized in conjunction with one or more conditions, constants, or boundary conditions (e.g., shape, dimensions, materials, . . . , etc.). The section below, entitled “Wave Spring Modeling”, provides a detailed description of this model, including the different variables available.

In the various embodiments of the invention, the spinal disc can include any type of spring design. The spring design can be adjusted to provide substantially matched stiffnesses of the native disc. In some embodiments of the invention, a spinal disc can be designed to have “generic” properties. Alternatively, a particular patient's biomechanical information can be collected and the spring design can be adjusted to provide a closer match than a generic design.

Although any spring design can be used for the spinal disc, a spring design can be identified to substantially match the sniffinesses of the native disc if a majority of the stiffness values for the spring are within 20-30% of the stiffiness values for the native disc. One such design has been found to be a multi-turn compression helical spring design, including wave spring and non-wave spring designs. A “wave” spring, as used herein, refers to a spring in which a nominally flat wire is formed in a helical shape and where the wire also has a substantially periodic sinusoidal pattern along the length of the wire. The amplitude and frequency of the waves in periodic pattern is provided such that adjacent waves in adjacent turns of the spring support each other, providing additional stiffness to the spring. That is, the adjacent turns are approximately 180° out of phase with each other. Wave springs also typically allow the stiffness of the spring to be further refined, namely through the adjustment of the amplitude and frequency of the sinusoidal wave pattern used to manufacture the spring. This allows refinements to be made for particular patients and materials being used. For example, the modeling section below shows typical wave spring geometry values obtained for particular sex, age, and weight groups and particular materials (17-4 stainless steel and Ti₆Al₄V).

Although wave springs provide spatially complaint springs with adjustable stiffness, some wave springs can still fail to provide a sufficiently stable joint. In some embodiments, the ends of the wave spring can be grounded or squared off. In other embodiments, an alternative wave spring geometry can be provided to support the ends of the springs and to prevent buckling, thus providing a more balanced and stable joint when implanted in a patient. In particular, a wave compression spring can be provided with a varying sinusoidal pattern over the length of the wire. That is, by including multiple portions of varying amplitudes, a wave spring can be provided in which the ends of the spring form substantially flat shims and are substantially perpendicular to the longitudinal axis of the spring.

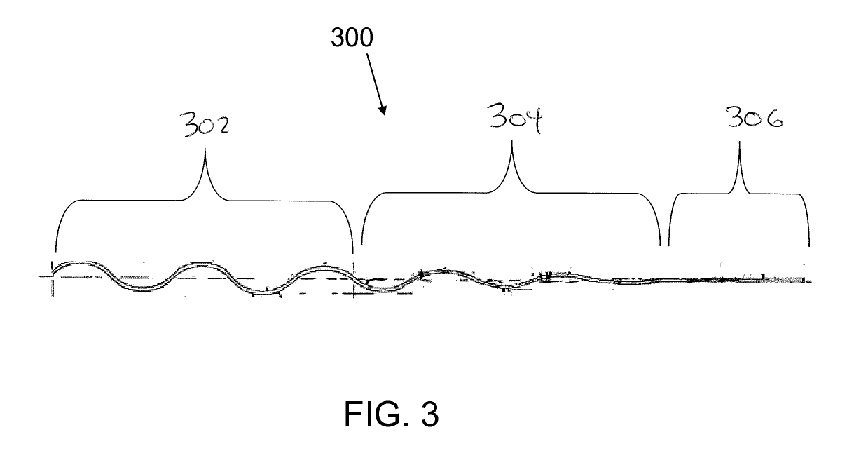

For example, as shown by the exemplary wire 300 in FIG. 3, the amplitude of the main body region 302 of the exemplary wire 300 is formed using a sinusoidal pattern having a fixed amplitude and frequency. The exact geometry of the main body region 302 can be selected to match or approximate the stiffnesses of the native disc, as previously described. Additionally, as previously described, the geometry can also be selected such that stresses in the spring remain under the endurance limit of the material used for forming the spring. To provide flat and stable ends, the amplitude of the sinusoidal pattern in the wire 300 is gradually reduced over the length of a transition region 304 so that a flat portion of the wire 300 can be formed in an end region 306. Accordingly, when the wire 300 is helically wound to form the wave spring, the end region 306 provides a substantially flat surface or shim at the end of the spring.

One of ordinary skill in the art will recognize that the invention is not limited solely to the sinusoidal pattern geometry shown in FIG. 3 and that it is within the scope of the invention to vary the length of the various regions of the wire. In particular, the lengths of the various regions 302, 304, 306 of the wire 300 can be varied to further adjust the geometry of a resulting spring to allow the designed to more closely match the stiffnesses of a native disc. Furthermore, the invention is not limited solely to the number of regions shown in FIG. 3. Multiple body regions, separated by transition regions, can be used to form multiple sinusoidal patterns for the spring. Alternatively, discrete regions of the sinusoidal pattern need not be formed and the geometry of a wire can vary continuously over the length of the wire.

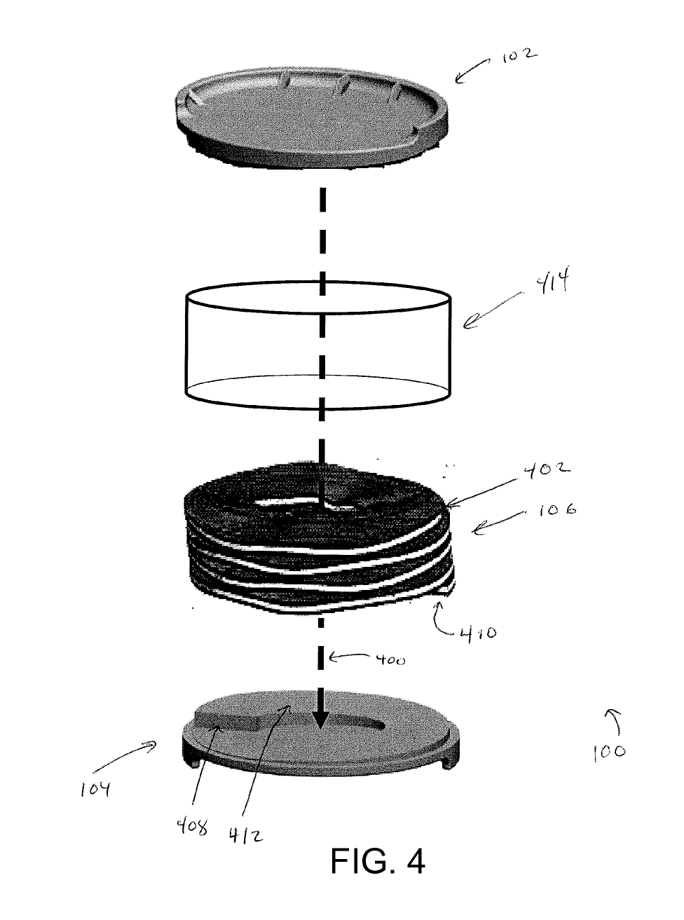

In addition to varying the geometry of the spring to provide stable ends, a geometry for the spring that is resistant to buckling can be provided using a wire that is substantially flat. That is, the wire used to form the spring has a cross-sectional width that is significantly greater than its cross-sectional height. This can be observed in the exploded view of disc 100, as shown in FIG. 4. The spring 106 can be formed using a flat wire 402 arranged in a helical pattern about a longitudinal axis 400. The present inventor has discovered that to ensure sufficient stability of the body of the spring, the flat wire can have a width greater than a height of the flat wire. Such a width to height ratio can substantially reduce the likelihood of buckling by decreasing the lateral flexibility of the wave spring. Furthermore, the increased contact surface area for adjacent waves in adjacent turns increases the overall stability of the spring and of the intervertebral prostheses.

As previously described, the spring 106 can be formed from steel or titanium alloys. However, in the various embodiments of the invention the spring 106 can be formed from a length of any substantially biocompatible metals, such as titanium, aluminum, iron, cobalt, chromium, and/or vanadium comprising alloys (e.g., titanium-aluminum-vanadium alloys, stainless steel, and cobalt-chromium alloys). In some embodiments, Ti₆Al₄V is used, as previous described. Ti₆Al₄V is a biocompatible material that has been used extensively for prosthetic implants. Accordingly, Ti₆Al₄V possesses a well-defined fatigue limit meaning that, if stresses can be kept below a certain design point (in this case ˜600 Mpa for completely reversed cyclic loading), an unlimited fatigue life can generally be assumed. This alloy also possesses a high strength to elastic modulus ratio (relative to other metals) which is the primary measure of material quality with respect to compliant mechanism design (i.e., a higher strength to modulus ratio implies that the given material can withstand larger deflections before failure). Finally, this titanium alloy may be used in modern near-net-shape manufacturing processes which allow for rapid and customizable production.

However, the invention is not limited to only metal comprising springs. In some embodiments, the spring 106 can be constructed biocompatible non-metals, such as polyethylene, polytetrafluoroethylene, certain carbon composites, or certain other polymer-comprising materials. It is also within the scope of the invention to coat or encapsulate the flat wire 402 using biocompatible materials. Similarly, the endplates 102, 104 can also be constructed from metal or non-metal biocompatible materials, as described above It is further within the scope of the invention to use non-biocompatible materials coated with biocompatible materials.

Furthermore, although the flat wire 402 can be formed using a single wire comprised of a single type of material, the invention is not limited in this regard. In some embodiments, the flat wire can be formed from a stack of different types of materials to fine tune the elastic properties of the spring. For example, a first material can be used to provide base characteristics for the spring 106, and one or more other materials can be used to counter or enhance the characteristics of the spring 106 to more closely match the stiffnesses of the natural disc. In another example, several layers of the same type of material can be used to form “strands” for the wire. In such embodiments, multiple strands provide increased flexibility for the spring. Accordingly, thickness and number of strands can be used in conjunction with the equations above to provide a further means for adjusting the properties of the spring being used. Using a multi-layer spring with Ti₆Al₄V and the models in the modeling section below, a design for a lumbar replacement disc can be obtained that provides stiffness values as shown below in Table 1:

TABLE I
PROPERTIES OF NATIVE, MODELED WAVE DISCS

| | | | Titanium |
|---|---|---|---|
| | | Native | Alloy |
| Type of | | Disc | Disc |
| Deformation | Units | Stiffness | Stiffness |
| Compression^{[1]} | MN/m | 2.3 | 2.3 |
| Shear^{[1]} | MN/m | 0.26 | 0.28 |
| Extension^{[2]} | Nm/deg | 1.3 | 0.93 |
| Flexion^{[2]} | Nm/deg | 0.8 | 0.93 |
| Lateral | Nm/deg | 1.1 | 0.93 |
| Bending^{[2]} | | | |
| Torsion^{[2]} | Nm/deg | 2 | 0.3 |

As seen in Table 1, the stiffness values of the titanium alloy disc are similar those of the native disc, with the exception of torsion. However, in the case of lumber replacement discs, matching torsion is typically not critical since torsion of lumbar discs is not a common event. Adjustment of the relative matching of stiffnesses can be accomplished by adding weights to the various parameters, signifying their importance during optimization, as described in the modeling section below. Therefore, in the example above for a lumbar replacement disc, stiffnesses other than torsion are weighted heavier. In contrast, for cervical discs, torsion would be weighted heavier, as it is a more common event.

Although the spring 106, as configured above, provides stability under a load, the spring 106 could still be displaced after implant in a patient if properly not retained in place. For example, under a load, the spring 106 could rotate or laterally shift. This can be due to the natural tendency of the ends of a spring to rotate under a load. Accordingly, in the various embodiments of the invention, the inner surface of the endplates 102, 104 can be configured to include one or more retaining structures to prevent such shifting. For example, as shown in FIG. 4, the lower endplate 104 and upper endplate (not shown) includes protrusions 408 extending from the inner surfaces of the endplates. The protrusions 408 can be designed so that when the spring 106 is compressed due to a load, the ends 410 of the spring 106 engage with the protrusion 408 as they attempt to rotate, holding the spring 106 in place. In some cases, as shown in FIG. 3, the protrusion 308 can include sloped regions 312 to improve contact with an end of the spring 106 and to transfer a load to the spring 106 more evenly.

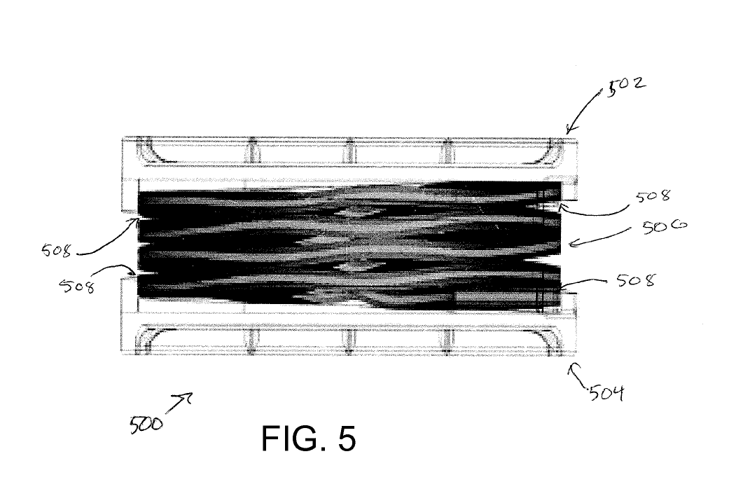

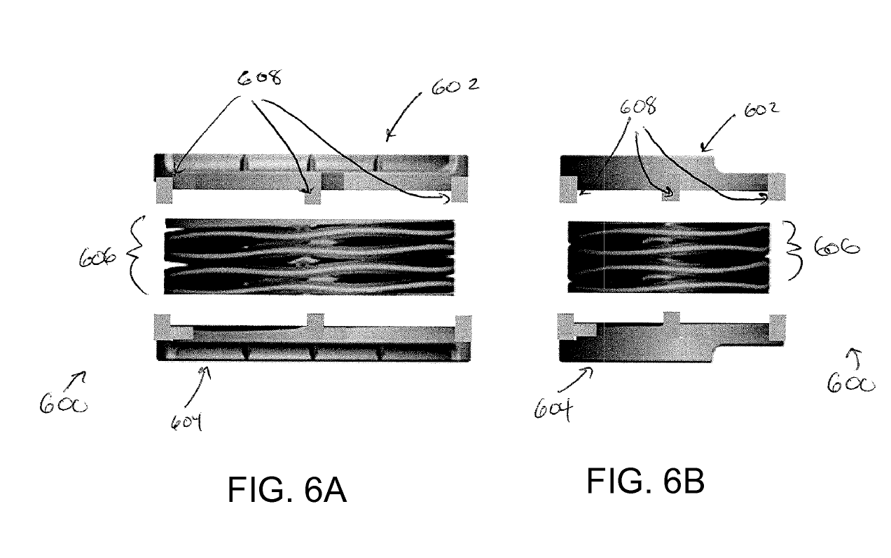

Additionally, the endplates can also include additional retaining features to prevent lateral motion. That is, to prevent a spring from shifting out from in between the endplates. In such embodiments, as shown in FIG. 5, a disc 500 can include endplates 502, 504 with one or more depressions 502 for the spring 506 to lie in and prevent lateral motion. Alternatively, as shown in FIGS. 6A and 6B, one or more protrusions 608 can extend from the endplates 602, 604, to prevent lateral motion of a spring 606 in the disc 600. It is also within the scope of the invention to include a combination of retaining structures in the various embodiments of the invention.

For a metal comprising spring and/or endplates, near-net-shape manufacturing processes (such as electron-beam melting and direct-metal laser sintering) can be utilized for fabrication which are capable of fabricating complex spatial geometries from biologically compatible metal alloys. The use of rapid manufacturing techniques enables a straightforward path for full customization of discs based on imaging data (e.g., magnetic resonance imaging or computerized tomography) for a specific patient, and in particular, with regard to the localized topology of the adjacent vertebral bodies, the height of the disc, the lordosis (i.e., relaxed curvature) of the spinal joint, and the compliance properties of the spring. Therefore, intervertebral discs according to the various embodiments of the invention can be fine tuned to the individual patient in terms of biomechanical and orthopedic requirements. This is in contrast to the relatively generic and discrete configurations available for conventional prosthetic discs.

As previously described, the spring and the endplates in the disc can be constructed from biocompatible materials. However any voids in the disc (such as the regions between adjacent waves in the disc) can still provide a path for growth of scar tissue and/or other tissues that can affect operation and/or life span of the disc. Accordingly, in some embodiments of the invention, the voids can be filled or covered with a low durometer biocompatible elastomer, such as medical grade silicone, which will not sustain any significant mechanical loads, but can prevent the growth of scar (and/or other) tissue into the voids of the device and will not otherwise impede its functionality. In one example, as shown in FIG. 4, an elastomer sheath 414 can be used to cover the spring 106. In another example, an elastomer material can be used to fill the voids. In yet another example, the spring 106 can be completely encapsulated by the elastomer and inserted as one piece between the endplates 102, 104. In these embodiments, an elastomer material having stiffness values significantly less than that of the spring 106 can be selected. This allows the stiffnesses of the spring to remain relatively unaltered. For example, the elastomer can have stiffnesses of 10% or less than the stiffnesses of the spring.

In the embodiments in FIGS. 1-6, the spring and the endplates are both generally elliptically shaped, as shown by the differences in width of the views in FIGS. 6A and 6B. The elliptical shape generally matches the natural shape of the native disc. Furthermore, the elliptical shape allows the disc to engage with vertebral bodies over a large surface area, while at the same time keeping the disc away from spinal nerves. This reduces the likelihood of damage to the spinal nerves during insertion or in the case of material accidental protruding from the disc. However, the invention is not limited to solely elliptical discs and any other shape for the spring and the discs can also be used. For example, a polygon-shaped spring can be used. Alternatively, an irregular shape, such as a kidney bean shape can also be used. However, for such alternate shapes, the amount of computation or difficultly of manufacture of the spring is increased.

### Wave Spring Modeling

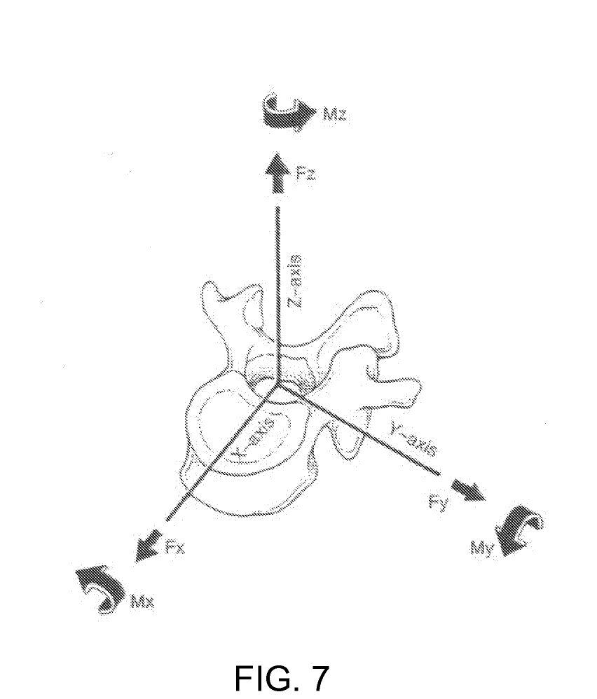

As described above, a mathematical model can be generated for providing a spring design that approximates, at least in part, the behavior of a native disc in the six degrees of freedom available for the native disc. These degrees of freedom with respect to reference axes for a vertebrate body are shown in FIG. 7. As shown in FIG. 7, the degrees of freedom define the forces experienced by a vertebrate body. These forces include moments with respect to each reference axes (Mx, My, Mz) and linear forces (Fx, Fy, Fz). Using these forces and the material properties for the proposed spring a model can be developed. For example titanium (Ti) and steel comprising materials typically have material properties:

Young's Modulus (E), [Pa] (˜120E9 Pa for Ti, ˜200E9 Pa for Steels) and

Shear Modulus (G), [Pa] (˜44.8E9 Pa for Ti, ˜76.9E9 Pa for Steels) where

$$G = \frac{E}{2 + 2 υ}$$

and where υ is Poisson's Ratio (˜0.34 for Ti, ˜0.30 for Steels).

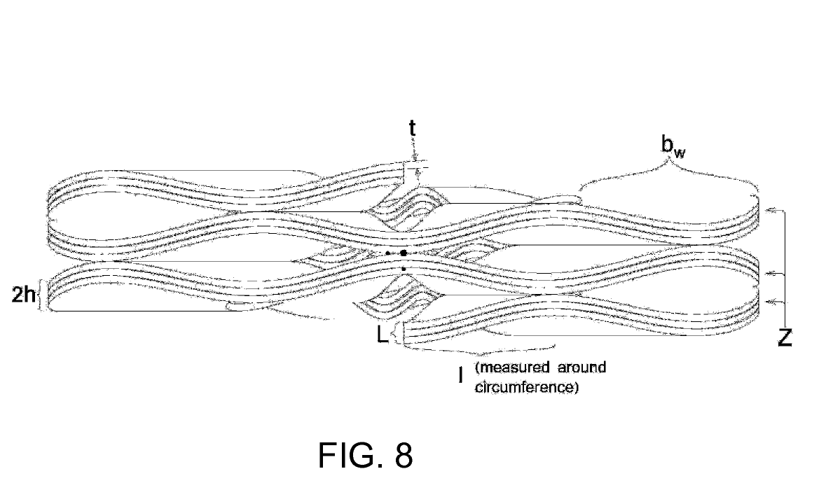

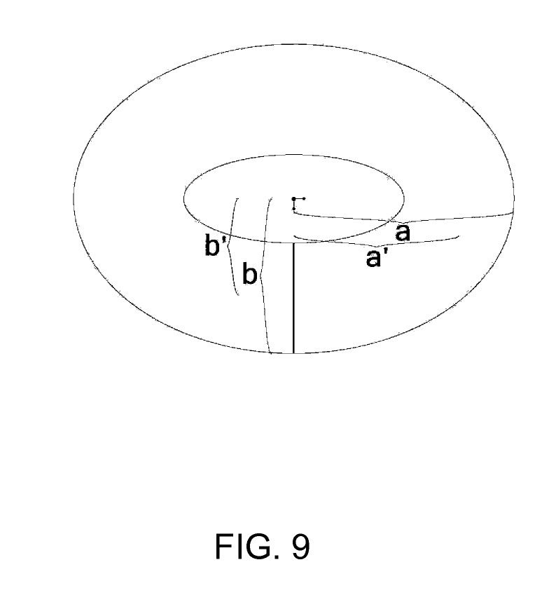

Assuming a multi-layer, multi-turn circular wave spring, as shown in FIGS. 8 and 9, the spring parameters for the model can be specified as follows:

L—Number of Layers

N—Number of Waves per Turn

Z—Number of Turns

The dimensional variables for the model can then be specified as:

Aᵢ—area of section i

a—ellipse major axis

a′—ellipse major axis minus half-width

b—ellipse minor axis

b′—ellipse minor axis minus half-width

b_{w}—cross-sectional width

e—eccentricity

h—peak amplitude (one-half peak to peak)

l—half wavelength

R—outer circular radius

\tilde{R}—outer circular approximation for ellipse

r—inner circular radius

\tilde{r}—inner circular approximation for ellipse

rₙ—radius of neutral axis

S—sum of squared distances

t—layer thickness

\tilde{Y}—Area centroid along y-axis for half circle or ellipse bisected by x-axis

y̅ᵢ—centroid ordinate of section i along y

X̅—Area centroid along x-axis for half circle or ellipse bisected by y-axis

x̅ᵢ—centroid ordinate of section i along x

These are shown in FIGS. 8 and 9 or derived therefrom.

For the model, the following constants can be defined:

α—virtual torsion-element constant, experimentally determined as

$$\frac{\sqrt{2}}{8} \approx .177$$

C—1.2 for rectangular cross-sections;

c₂—torsional constant, as given below in Table 2:

TABLE 2
TORSIONAL CONSTANTS

| b/t | c₂ |
|---|---|
| 1 | 0.1406 |
| 1.2 | 0.1661 |
| 1.5 | 0.1958 |
| 2 | 0.229 |
| 2.5 | 0.249 |
| 3 | 0.263 |
| 4 | 0.281 |
| 5 | 0.291 |
| 10 | 0.312 |
| inf | 0.333 |

The total stiffnesses in the case of a circular wave spring can then be modeled using models for axial, bending, shear, and torsional stiffnesses. The model of axial stiffness given by:

$${K}_{axial} = {[ \frac{1}{2 E} \frac{{l}^{2} \sqrt{4 {h}^{2} + {l}^{2}}}{{b}_{w} {t}^{3}} ]}^{- 1} ( \frac{NL}{Z} ) .$$

The model for bending stiffness is given by:

$${K}_{bending} = \frac{{( \overline{Y} )}^{2}}{2} {K}_{axial} where \overline{Y} = \frac{4}{3 π} ( \frac{{R}^{2} + Rr + {r}^{2}}{R + r} ) .$$

The model for shear stiffness is given by:

$${K}_{shear} = {( \frac{1}{{K}_{shear , i n \text{-} plane}} + \frac{1}{{K}_{shear , out \text{-} plane}} )}^{- 1} ,$$

where in-plane shear stiffness is given by:

$${K}_{shear , i n \text{-} plane} = {[ \frac{π {R}_{m}}{4 {b}_{w} t} ( \frac{{R}_{m}}{eE} - \frac{1}{E} + \frac{C}{G} ) ]}^{- 1} ( \frac{L}{Z} ) , \\ {R}_{m} = \frac{R + r}{2} , and e = {R}_{m} - {r}_{n} , {r}_{n} = \frac{{b}_{w}}{ln \frac{R}{r}} ,$$

and where out of plane shear stiffness is given by:

$${K}_{shear , out \text{-} plane} = {\frac{{Gc}_{2} {b}_{w} {t}^{3}}{α ( 2 h ) {R}_{m}^{2}} [ {\sum }_{i = 1}^{2 N} {( sin ( \frac{π}{Z} ( 2 i - 1 ) ) )}^{2} ]}^{- 1} ( \frac{L}{Z} ) .$$

The model for torsional stiffness is given by:

$${K}_{torsion} = {( \frac{1}{{K}_{torsion , i n \text{-} plane}} + \frac{1}{{K}_{torsion , out \text{-} plane}} )}^{- 1} ,$$

where in-plane torsional stiffness is given by:

$${K}_{torsion , i n \text{-} plane} = \frac{{Eb}_{w}^{3} t}{24 π {R}_{m}} ( \frac{L}{Z} ) ,$$

and where out of plane shear stiffness is given by:

$${K}_{torsion , out \text{-} plane} = \frac{{Gc}_{2} {b}_{w} {t}^{3}}{α ( 2 h )} ( \frac{L}{2 NZ} ) .$$

The total stiffnesses in the case of an elliptical wave spring can be modeled similarly using models for axial, bending, shear, and torsional stiffnesses. In the case of an elliptical wave spring, the model of axial stiffness is given by:

$${K}_{axial} = {[ \frac{1}{2 E} \frac{{l}^{2} \sqrt{4 {h}^{2} + {l}^{2}}}{{b}_{w} {t}^{3}} ]}^{- 1} ( \frac{NL}{Z} ) .$$

The model for bending stiffness includes a lateral being stiffness:

$${K}_{bending , lateral} = \frac{{( \overline{Y} )}^{2}}{2} {K}_{axial} where \overline{Y} = \frac{\sum {A}_{i} {\overline{y}}_{l}}{\sum {A}_{i}} , \\ {A}_{i} = \frac{1}{2} π {a}_{i} {b}_{i} , {\overline{y}}_{i} = \frac{4 {a}_{i}}{3 π}$$

and a flexion-extension bending stiffness:

$${K}_{bending , flexion \text{-} extension} = \frac{( \overline{X} )}{2} {K}_{axial} where \overline{X} = \frac{\sum {A}_{i} {\overline{x}}_{i}}{\sum {A}_{i}} , \\ {A}_{i} = \frac{1}{2} π {a}_{i} {b}_{i} , {\overline{x}}_{i} = \frac{4 {b}_{i}}{3 π}$$

In the expressions above for bending stiffness, i ε{1,2} where 1 and 2 denote the outer (larger) ellipse and the inner (smaller) ellipse, respectively, as seen when looking down on the spring from above, such as in FIG. 9.

The model for shear stiffness includes a lateral shear stiffness:

$${K}_{shear , lateral} = {( \frac{1}{{K}_{shear , i n \text{-} plane , lateral}} + \frac{1}{{K}_{shear , out \text{-} plane , lateral}} )}^{- 1} , \\ where$$

$${K}_{shear , i n \text{-} plane , lateral} = {[ \frac{π {R}_{m}}{4 {b}_{w} t} ( \frac{{R}_{m}}{eE} - \frac{1}{E} + \frac{C}{G} ) ]}^{- 1} ( \frac{L}{Z} ) , \\ {R}_{m} = \frac{\tilde{R} + \tilde{r}}{2} , \tilde{R} = a , \tilde{r} = b - {b}_{w} , e = {R}_{m} - {r}_{n} , \\ and {r}_{n} = \frac{{b}_{w}}{ln \frac{\tilde{R}}{\tilde{r}}} ;$$

$$and where$$

$${K}_{shear , out \text{-} plane , lateral} = \frac{{Gc}_{2} {b}_{w} {t}^{3}}{α ( 2 h ) S} ( \frac{L}{Z} ) , \\ S = {\sum }_{i = 1}^{2 N} [ {( \frac{{a}^{′} {b}^{′}}{\sqrt{\begin{matrix}{( {a}^{′} )}^{2} {( sin ( \frac{π}{Z} ( 2 i - 1 ) ) )}^{2} + \\ {( {b}^{′} )}^{2} {( cos ( \frac{π}{Z} ( 2 i - 1 ) ) )}^{2}\end{matrix}}} )}^{2} {( sin ( \frac{π}{Z} ( 2 i - 1 ) ) )}^{2} ] , \\ and {a}^{′} = a - \frac{{b}_{w}}{2} , {b}^{′} = b - \frac{{b}_{w}}{2}$$

The model for shear stiffness also includes an antero-posterior shear stiffness:

$${K}_{shear , antero \text{-} posterior} = {( \frac{1}{{K}_{shear , i n \text{-} plane}} + \frac{1}{{K}_{shear , out \text{-} plane , antero \text{-} posterior}} )}^{- 1}$$

$$where$$

$${K}_{shear , in \text{-} plane , antero \text{-} posterior} = {[ \frac{π {R}_{m}}{4 {b}_{w} t} ( \frac{{R}_{m}}{eE} + \frac{1}{E} + \frac{C}{G} ) ]}^{- 1} ( \frac{L}{Z} ) , {R}_{m} = \frac{\tilde{R} + \tilde{r}}{2} , \\ \tilde{R} = a , \tilde{r} = b - {b}_{w} , e = {R}_{m} - {r}_{n} , \\ and {r}_{n} = \frac{{b}_{w}}{ln \frac{\tilde{R}}{\tilde{r}}} , \\ and where$$

$${K}_{shear , out \text{-} plane , anter \text{-} posterior} = \frac{{Gc}_{2} {b}_{w} {t}^{3}}{α ( 2 h ) S} ( \frac{L}{Z} ) , \\ S = {\sum }_{i = 1}^{2 N} {[ ( a - \frac{{b}_{w}}{2} ) - ( \frac{{a}^{′} {b}^{′}}{\sqrt{\begin{matrix}{( {a}^{′} )}^{2} {( sin ( \frac{π}{Z} ( 2 i - 1 ) ) )}^{2} + \\ {( {b}^{′} )}^{2} {( cos ( \frac{π}{Z} ( 2 i - 1 ) ) )}^{2}\end{matrix}}} ) ( cos ( \frac{π}{Z} ( 2 i - 1 ) ) ) ]}^{2} , \\ {a}^{′} = a - \frac{{b}_{w}}{2} , and {b}^{′} = b - \frac{{b}_{w}}{2} .$$

The model for torsional stiffness is given by:

$${K}_{torsion} = {( \frac{1}{{K}_{torsion , in \text{-} plane}} + \frac{1}{{K}_{torsion , out \text{-} plane}} )}^{- 1} ,$$

where in-plane torsional stiffness is given by:

$${K}_{torsion , in \text{-} plane} = \frac{{Eb}_{w}^{3} t}{24 π {R}_{m}} ( \frac{L}{Z} ) ,$$

and where out of plane shear stiffness is given by:

$${K}_{torsion , out \text{-} plane} = \frac{{Gc}_{2} {b}_{w} {t}^{3}}{α ( 2 h )} ( \frac{L}{2 NZ} ) .$$

These models can be used to generate an optimization algorithm which, based on a desired Axial Stress, varies the number of turns (Z), waves per turn (N), number of layers (L), wave height (h), and thickness (t) so that it may return the lowest stress design (combination of parameters). This stress can be calculated according to the following equation

$$σ ( stress ) = \frac{My}{I} = \frac{3 Fl}{2 {b}_{w} {t}^{3}} \frac{1}{LN}$$

During this process, the length (l) and cross-sectional width (b_{w}) can be solved for as they are constrained by other variables. As a result, such an algorithm can return the lowest stress design for a given load and axial stiffness.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Using known ranges of body mass, walking loads, vertebral widths, vertebral depths, disc heights and by selecting a disc height and a desired axial stiffness, nominal design values can be obtained for various types of patients. However, the mean age of patients undergoing spinal fusion procedures is generally 50-59 years. For exemplary purposes, the results of designs based on the 10^{th}, 50^{th}, and 90^{th}, Body Mass Percentiles for both Males and Females of this age group are presented below along with the design parameter ranges they imply. Although the resulting ranges for selected design parameters are shown below, a much larger design space (larger range of parameters) is typically searched to obtain nominal designs. Typically, the lumbar spine endures compressive forces of 1.0-2.5 times body weight during normal level walking. Accordingly, this values has been used as a means of determining the maximum walking load and is assumed to be representative of the maximum cyclical load the spine should endure during daily activities. For purpose of design, it has also been assumed that the desired axial stiffness varies linearly in proportion to body mass, and that a value of 2.3 MN/m is representative of the 50^{th} percentile of the population. The constraints on Upper Vertebral Width (which constrains a, the ellipse major axis), Upper Vertebral Depth (which constrains b, the ellipse minor axis), and Disc Height (constrains total design height) are based on the typical geometrical dimensions of the lower lumbar vertebrae.

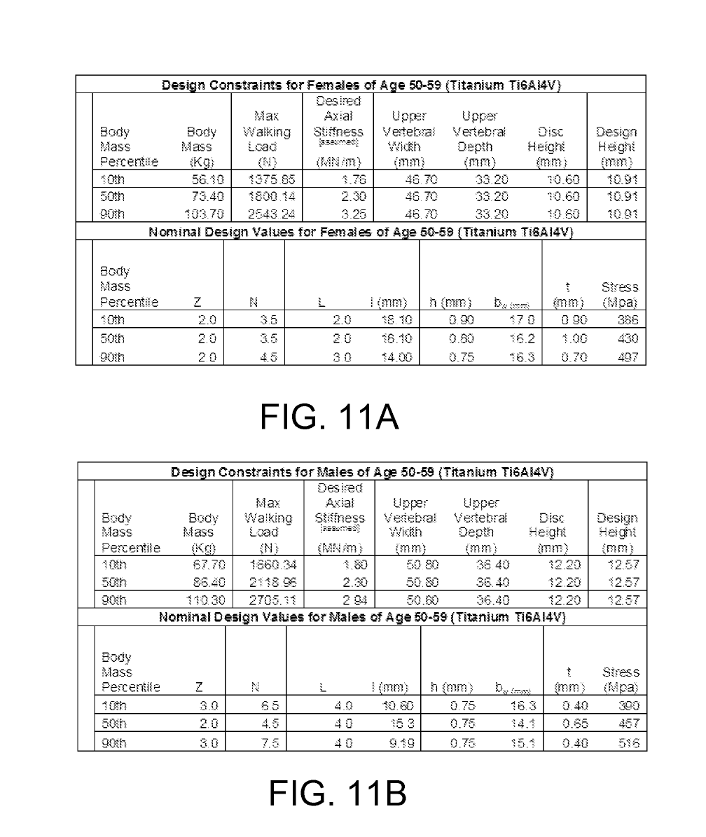

Typical resulting values from the models and algorithm discussed above are shown in FIGS. 10A and 10B for 17-4 stainless steel elliptical wave spring designs. FIGS. 10A and 10B tabulate design constraints and nominal design values for female and male patients, respectively, in the age group 50-59. FIGS. 11A and 11B shown these values Ti₆Al₄V elliptical wave spring designs. FIGS. 11A and 11B tabulate design constraints and nominal design values for female and male patients, respectively, in the age group 50-59. FIGS. 10A, 10B, 11A, and 11B are presented by way of example and not limitation. However, the invention is not limited to this age group, the materials shown, or the design parameters shown in these figures. As a result, the design parameters can vary. For example, design ranges as shown below in Table 3 for titanium and steel comprising prostheses:

TABLE 3
EXEMPLARY RANGE OF DESIGN PARAMETERS FOR
TITANIUM AND/OR STEEL-COMPRISING PROSTHESIS

| Z | N | L | l (mm) | h (mm) | b_{w (mm)} | t (mm) | Stress (Mpa) |
|---|---|---|---|---|---|---|---|
| 2-3 | 3.5-7.5 | 2-4 | 10.6-19.7 | 0.75-0.90 | 15.1-17.0 | 0.40-1.00 | 386-647 |

Applicants present certain theoretical aspects above that are believed to be accurate that appear to explain observations made regarding embodiments of the invention. However, embodiments of the invention may be practiced without the theoretical aspects presented. Moreover, the theoretical aspects are presented with the understanding that Applicants do not seek to be bound by the theory presented.

While various embodiments of the invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has” “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the following claims.

What is claimed is:

1. A prosthesis for replacing a native disc between first and second adjacent vertebral bodies, comprising:
a compliant element having a first composition and a geometry for providing a plurality of element stiffnesses for said compliant element substantially matching spatial stiffnesses of said native disc;
an upper plate of said first or a second composition, said upper plate having opposed inner and outer surfaces, said upper plate inner surface having a first retaining structure for affixing a position of said first end of said compliant element; and
a lower plate of said first or a second composition, said lower plate having opposed inner and outer surfaces, said lower plate inner surface having a second retaining structure for affixing a position of said second end of said compliant element.

2. The prosthesis of claim 1, wherein said first composition comprises at least one metal.

3. The prosthesis of claim 2, wherein said spatially compliant element has an endurance limit greater than the load to which it is subjected under nominal native disc loading conditions;

4. The prosthesis of claim 2, wherein said spatially compliant element comprises: a wave spring formed from a flat wire helically wound about a longitudinal axis.

5. The prosthesis of claim 4, wherein said flat wire is helically wound in an elliptical path about said longitudinal axis.

6. The prosthesis of claim 4, wherein said wave spring has between 2 and 5 turns and between 3 and 10 waves per turn.

7. The prosthesis of claim 6, wherein a crest-to-crest height of said waves is between 0.15 and 1.5 mm, and wherein a wavelength for said waves is between 20 and 50 mm.

8. The prosthesis of claim 7, wherein said crest-to-crest height in at least a portion of said first and said second ends is less than a crest-to-crest height in said other spring portions.

9. The prosthesis of claim 4, wherein said flat wire has a width between 10 and 20 mm.

10. The prosthesis of claim 4, wherein said flat wire comprises a plurality of layers.

11. The prosthesis of claim 10, wherein said flat wire has between 2 and 5 layers.

12. The prosthesis of claim 10, wherein a height of each of said layers is between 0.3 and 1.5 mm.

13. The prosthesis of claim 4, wherein at least a portion of said first and said second compositions are biocompatible.

14. The prosthesis of claim 2, further comprising:
one or more biocompatible elastomer portions filling one or more void regions within said spatially compliant element or between said upper plate and said lower plate, said elastomer portions having sniffinesses less than said plurality of element stiffnesses.

15. The prosthesis of claim 2, further comprising one or more biocompatible coatings covering at least one among said spatially compliant element, said upper plate, and said lower plate.

16. A method for designing a prosthesis for replacing a native disc between first and second adjacent vertebral bodies, comprising:
determining a geometry for a spatially compliant element of a first composition, said geometry providing a plurality of element stiffnesses for said spatially compliant element substantially matching spatial stiffnesses of said native disc, said geometry distributing a force applied to at least one of a first and a second end of said spatially compliant element to a plurality of other portions of said spatially compliant element such that a portion of said force distributed to each of said other spatially compliant element portions under nominal native disc loading conditions is less than an endurance limit of said first composition;
designing an upper plate of said first or a second composition, said upper plate having opposed inner and outer surfaces, said upper plate inner surface designed to have a first retaining structure for affixing a position of said first end of said spatially compliant element; and
designing a lower plate of said first or a second composition, said lower plate having opposed inner and outer surfaces, said lower plate inner surface designed to have a second retaining structure for affixing a position of said second end of said spatially compliant element.

17. The method of claim 16, wherein said geometry comprises a wave spring geometry using a flat wire wound about a longitudinal axis.

18. The method of claim 17, wherein said fiat wire is wound in an elliptical path.

19. The method of claim 17, wherein said wave spring geometry has between 2 and 5 turns and between 3 and 10 waves per turn.

20. The method of claim 19, wherein a crest-to-crest height of said waves is selected to be between 0.15 and 1.5 mm, and wherein a wavelength for said waves is selected to be between 20 and 50 mm.

21. The method of claim 20, wherein said crest-to-crest height in at least a portion of said first and said second ends is selected to be less than a crest-to-crest height in said other spring portions.

22. The method of claim 17, wherein a width of said flat wire is selected to be between 10 and 20 mm.

23. The method of claim 17, wherein said flat wire is designed to include a plurality of layers.

24. The method of claim 23, wherein a height of each of said layers is selected to be between 0.3 and 1.5 mm.

25. A prosthesis for replacing a native disc between first and second adjacent vertebral bodies, comprising:
a wave spring having a first composition and a geometry for providing stiffnesses for said spring substantially matching a stiffnesses of said native disc, said geometry distributing a force applied to at least one of a first and a second end of said spring to a plurality of other portions of said spring, and said first composition having an endurance limit greater than a portion of said force distributed to each of said other spring portions under nominal native disc loading conditions;
an upper plate of said first or a second composition, said upper plate having opposed inner and outer surfaces, said upper plate inner surface having a first retaining structure for affixing a position of said first end of said spring; and
a lower plate of said first or a second composition, said lower plate having opposed inner and outer surfaces, said lower plate inner surface having a second retaining structure for affixing a position of said second end of said spring.
